(19) United States
(12) Patent Application Publication
(10) Pub. No.: US 2003/0032672 A1
(43) Pub. Date: Feb. 13, 2003

(54) Method for preparing a conjugated linoleic acid-containing structured lipid and use of the same

(76) Inventors: In Hwan Kim, Seoul (KR); Soo Hyun Chung, Seoul (KR); Jae Hong Jeong, Kyunggi-do (KR); Chil Surk Yoon, Seoul (KR)

Correspondence Address: MARTINE & PENILLA, LLP, 710 LAKEWAY DRIVE, SUITE 170, SUNNYVALE, CA, 94085, US

(21) Appl. No.: 10/126,093

(22) Filed: Apr. 18, 2002

(30) Foreign Application Priority Data
Apr. 19, 2001 (KR) 2001-21201

(51) Int. Cl. A61K 31/231; C07C 59/147; C07C 59/185

(52) U.S. Cl. 514/547; 554/121

(57) ABSTRACT

Disclosed is a method for preparing a CLA-enriched structured lipid by a transesterification of medium chain triglyceride (MCT) with ester or free fatty acid form of CLA using lipases, and use of the same. It has been known that CLA mainly exists in an acid form, and has various beneficial biological activities, but rapid oxidation property during storage. And also, animal fat or plant oil, widely ingested by animals or humans, is naturally produced as an acylglycerol form containing various fatty acids. The CLA-containing structured lipid is manufactured by mixing free or ester form of CLA with acylglyceride at a molar ratio of 1:1˜1:5, adding immobilized lipase of 2.2˜20% by weight of CLA and acylglyceride to the mixture with a solvent, and incubating at 35˜75° C. for 1-36 hours. The CLA-containing structured lipid is a natural TG form and contains a high content of CLA, characterized by at least 15% content of CLA in total fatty acid composition, and at least 5% of CLA at sn-2 position. Accordingly, with the CLA-enriched MCT being administrated, it can efficiently provide biological activities of CLA, such as inhibition of carcinogenesis and reduction of fat accumulation in a body, as well as rapidly supply calories, an innate nutritional property of MCT.

### TECHNICAL FIELD

The present invention relates to a method for preparing a conjugated linoleic acid (hereinafter, referred to as “CLA”)-enriched structured lipid by transesterification of medium chain triglyceride with CLA in the presence of lipases, and use of the same. More particularly, the present invention is concerned with cis-9, trans-11 octadecadienoic acid or trans-10, cis-12 octadecadienoic acid which has physiological activity.

### PRIOR ART

CLA is a general nomenclature for a positional and geometric isomer of linoleic acid having conjugated double bonds with cis or trans configuration.

It has been known that CLA has the nutritional and physiological activity of inhibiting mutations and inhibiting or reducing the occurrence of cancer in the skin, the stomach, the breast, and the large intestine. Also, CLA is known to relieve arteriosclerosis, treat diabetes by reducing sensitivity to glucose, and prevent obesity by reducing body fat.

CLA is naturally present in small amount as an acylglyceride form in beef and dairy products of ruminants. CLA can be prepared in fatty acid forms on the basis of alkali-isomerization reaction, as disclosed in Korean Pat. Application No. 99-11774, Canadian Pat. No. 2,219,601 (1997), nd U.S. Pat. No. 5,554,646 (1996). However, most of the fatty acids found in foods for animals or humans are not in free fatty acid forms, but in phosphoglyceride or acylglyceride forms. In addition, acylglyceride and free fatty acid forms follow different metabolic pathways in the body. Especially, the free fatty acid is relatively less preferable owing to its bad taste on ingestion and a more rapidly increased rancidity during storage. Thus, CLA in fatty acid form suffers the same problems.

Structured lipid (SL) is a new form of lipid made from the artificial linkage of specific fatty acids to triglyceride (TG) using chemical catalysts or enzymes. Typically, physiologically active fatty acids, such as an eicosapentaenoic acid (EPA), a docosahexaenoic acid (DHA), linolenic acid, dihomogamma-linolenic acid, behenic acid, stearic acid and the like, are linked to short chain triglycerides or medium chain triglycerides, followed by modification to improve the physiological activity of the fatty acid. For example, SL may be used as a low-caloric ingredient or may be enriched in various biological functions by containing natural ω-6 or ω-3 fatty acid, as well as being lowered in oxidation velocity and improved in body absorption rate.

Various methods for preparing structured lipids are known. Mechanical mixing of triglycerides different in fatty acid composition was introduced, but now, is not used because the triglyceride components are not compatible, but are seprated from each other owing to the different fatty acid composition. Instead, a chemical method using catalysts has been generally used, and is disclosed in U.S. Pat. Nos. 5,288,512; 5,662,953; 4,847,296; 4,871,768; 5,312,836; 4,528,197; 5,986,119; 5,571,553; and 6,013,665, and WO 00/15043. In these patents, structured lipids are made from chemical interesterification of fatty acids or fats of interest with fatty acids linked to a glycerol backbone, such as short chain triglyceride, medium chain triglyceride or natural lipids having different fatty acids. On the whole, this chemical interesterification is conducted at a reaction temperature of 150˜250° C. for 0.5˜4.0 hours, using a catalyst, such as sodium methylate, sodium methoxide, methane sulfonic acid, stannous oxylate, and the like, at an amount of 0.05˜0.5% by weight of the reaction substrate. But, this chemical method has disadvantages that secondary by-products such as lipid oxides are additionally produced under the high temperature condition, and the content of fatty acids of interest to be incorporated into triglycerides at specific positions (e.g., sn-2 position) is difficult to control because added fatty acids or fatty acids freed from their natural position in the triglyceride, as shown in the following structure, are randomly rearranged to R₁, R₂, and R₃ positions of the triglyceride in the catalysis reaction. Besides, there is still not known a method for preparing a structured lipid with CLA incorporated by a chemical method.

[Structure of Triglyceride]

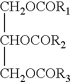

On the other hand, enzymatic methods in which SL is synthesized through transesterification by use of a lipase catalyzing hydrolysis of glyceride, have the advantage of being capable of selectively incorporating fatty acids to a specific position of triglyceride, conducting the synthesis at lower temperatures than the chemical synthesis method, producing almost no undesirable secondary by-products, and allowing the reactions TG+TG, TG+fatty acid, TG+fatty acid ester, and glycerol+fatty acid to be performed. For the enzymatic synthesis, medium chain triglyceride (MCT) is used as a glycerol backbone. Relevant methods may be found in U.S. Pat. Nos. 5,219,744, 5,908,654 and 5,288,619, in which SL is prepared from plant oil, fish oil and/or stearic acid with glycerol used as a backbone.

Use of CLA in synthesizing SL is disclosed in U.S. Pat. No. 6,159,525 and WO 00/18944. U.S. Pat. No. 6,159,525 describes an edible fat spread such as margarine and butter containing CLA, wherein the fat of the fat spread comprises triglycerides of fatty acid residues, 0.05˜20% by weight of which consist of conjugated linoleic acid (CLA) residues, prepared by adding Mucor miehei lipase to a mixture of a CLA and palm oil (weight ratio 3:10). WO 00/18944 describes an acylglyceride having CLA of 50˜100% by weight, prepared by using Candida antarctica lipase and a reaction substate consisting of glycerol and CLA.

However, problems are found in the CLA-containing structured lipids manufactured according to the above methods, as follows. 1. The SL is low in CLA content. 2. Although the total content of CLA is high, contents of CLA at sn-1, -2 and -3 position of triglyceride, especially sn-2 position, are not defined, and also cannot be controlled.

The incorporated position of CLA in a triglyceride is important for its physiological activity. When an animal or human ingests triglyceride, pancreatic lipase catalyzes cleavage of fatty acids from sn-1 or 3 positions of triglyceride, and the free fatty acids thus formed are rapidly consumed by selective hydrolysis of lipoprotein lipases, resulting in energy release.

On the other hand, sn-2 monoglyceride is not easily hydrolyzed by a pancreatic lipase, and accordingly, it can stay for a long time in the intestine, resulting in high efficiency of absorption. Fatty acids of the absorbed sn-2 monoglyceride are incorporated into chylomicrons, and carried to a specific tissue, such as the liver or peripheral tissue, through the bloodstream and lymphatic system. Accordingly, the fatty acids of the absorbed sn-2 monoglyceride can perform its physiological functions in specific tissues without loss of biological activities by oxidation. Therefore, in case of the synthesis of structured lipid (SL), it is most preferable that specific fatty acids are selectively incorporated at the sn-2 position of the triglyceride.

### DISCLOSURE OF THE INVENTION

To overcome the above problems, it is an object of the present invention to provide a method for preparing a CLA-containing structured lipid in triglyceride form by adding immobilized lipase to a reaction composition comprising medium chain trigylceride (MCT) as a glycerol backbone, which consisting of medium chain fatty acids such as caproic acid (C_{6:0}), caprylic acid (C_{8:0}) and capric acid (C_{10:0}), and acid or ester forms of CLA, and controlling reaction conditions, and particularly, to provide a method for preparing a structured lipid enriched in CLA at sn2 position.

Additionally, upon ingesting a CLA-containing MCT of the present invention, high calorie value of MCT can be rapidly supplied, and besides, physiological benefits of CLA such as inhibition of carcinogenesis or reduction of fat accumulation in a body can be supplied.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a graph showing changes in tumor volume of mammary cancer with time; and

FIG. 2 is a graph showing changes in incidence of stomach cancer as determined by histopathological examination.

### BEST MODES FOR CARRYING OUT THE INVENTION

In the present invention, a CLA-enriched medium chain triglyceride is prepared by reacting a mixture comprising MCT as a glycerol backbone and an acid or ester form of CLA on an immobilized phase of a lipase under optimal conditions in terms of molar ratio of MCT and CLA, enzyme amount, reaction time and temperature. The CLA-containing MCT prepared according to the present invention contains CLA at a content of at least 15% by weight in total, with CLA linked to sn-2 position amounting to at least 5% of the total fatty acids.

Examples of the immobilized lipases used in the present invention include lipase LAK (Pseudomonas fluorecens), lipase AYS (Candida rugosa), lipase AS (Aspergillus niger) and lipase PS-C (Pseudomonas cepacia), which were purchased from Amano Pharmaceutical Co., Ltd. (Nagoya, Japan), and lipase IM (Rhizomucor miebei) and novozyme 435 (Candida antartica), which were purchased from Novo Nordisk Bioindustry Ltd. (Seoul, Korea), and a pancreatic lipase which was purchased from Sigma Chemical Company (St. Louis, Mo.). Useful is tricaprylin as an MCT, a product of Sigma Chemical Company, and CLA-ethyl ester (purity 99%) and a fatty acid form of CLA (purity 99%), purchased from Nu-Chek-Prep, Inc. (Elysian, Minn.).

A CLA-containing MCT of the present invention is an acylglyceride with biological activities, and its structure is represented by the general formula I.

[General formula I]

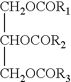

wherein R₁, R₂ and R₃, which may be the same or different, are akyl groups selected from the group consisting of alkyl moieties of medium chain fatty acids of C₆ to C₁₀ and long chain fatty acids of C_{18:2,} said C_{18:2} being a conjugated linoleic acid (CLA) selected from among cis-9, trans-11 octadecadienoic acid and trans-10, cis-12 octadecadienoic acid.

Useful in the present invention is a medium chain fatty acid selected from the group consisting of caproic acid (C_{6:0}), caprylic acid (C_{8:0}), capric acid (C_{10:0}), and mixtures thereof. The SL of the present invention contains cis-9, trans-11 or trans-10, cis-12 octadecadienoic acid in an amount of at least 15% by weight of the total composition of fatty acids located to the R₁, R₂ and R₃. In addition, cis-9, trans-11 or trans-10, cis-12 octadecadienoic acids at sn-2 position amounts to at least 5% of the total fatty acid.

In an embodiment of the present invention, CLA-containing SL is prepared by mixing free or ester forms of CLA with acylglyceride at a molar ratio of 1:1˜1:5, adding an immobilized lipase in an amount of 2.2˜20% by weight of the mixture of CLA and acylglyceride, along with a solvent, and reacting at 35˜75° C. for 1˜36 hours.

The solvent used in the enzyme reaction is selected from the group consisting of isooctane, n-hexane, pentane, toluene, benzene, chloroform, acetone and mixtures thereof.

The CLA-enriched SL, as prepared above, is useful for feedstuffs, foods, and medical treatment, with biological activities of inhibition of carcinogenesis and reduction of fat accumulation in the body as well as rapid calorie supply, and preferably, feedstuff or food contains the CLA-enriched structure lipid at least 0.1% by weight.

The present invention will be explained in more detail with reference to the following examples in conjunction with the accompanying drawings. However, the following examples are provided only to illustrate the present invention, and the present invention is not limited to them.

### EXAMPLE 1

### Preparation of CLA-Enriched Structured Lipids

A mixture of tricaprylin as MCT and an ester form or a free fatty acid form of CLA in a molar ratio of 1:1˜1:5 was charged, along with lipase at 2.5˜20%, and 3 ml of n-hexane as a solvent, into a cap-tube, incubated for 1˜36 hours in a shaking water bath at 35˜75° C., and then filtered. The resulting reaction product was separated by TLC to obtain triglyceride (TG) portion. Part of the TG portion was used for analysis of total fatty acid composition, and the rest was used for analysis of fatty acid composition at sn-2 position of the synthesized TG and CLA content was also measured. For the analysis of fatty acid composition at sn-2 position, pancreatic lipase was used and the analysis was carried out according to the method of Luddy et al. (Luddy et al., Journal of American Oil Chemists Society. 41. 693-696, 1964), and an optimal reaction condition, in which MCT having CLA located at sn-2 position could be obtained at a high content, was determined.

An examination was made of CLA contents of CLA-containing MCT according to lipases, according to reaction solvents, according to mix ratios of MCT and ester form of CLA, according to contents of lipase enzyme, according to reaction times, and according to reaction temperatures, and the results are shown in Table 1 to 6, respectively.

TABLE 1
CLA content of CLA-containing MCT according to lipases

| | Enzyme | | | | | |
|---|---|---|---|---|---|---|
| | Novozym | Lipozym | Lipase | Lipase | Lipase | Lipase |
| | 435 | IM | PS-C | LAK | AYS | AS |
| CLA | 56.9 ± 1.5 | 53.8 ± 0.4 | 41.4 ± 2.5 | 6.2 ± 0.8 | 0.9 ± 0.1 | 1.1 ± 0.2 |
| content | | | | | | |
| A (%) | | | | | | |
| CLA | 55.2 ± 2.4 | 54.5 ± 1.3 | 32.0 ± 1.1 | 7.8 ± 0.4 | 1.2 ± 0.3 | 0.8 ± 0.1 |
| content | | | | | | |
| B (%) | | | | | | |

Note: A: Ester form of CLA,
B: Free fatty acid form of CLA.
* A molar ratio of tricaprylin and CLA is 1:2 by weight, and the reaction was performed at 55° C. for 24 hours with 10% enzyme in hexane.

TABLE 2
CLA content of CLA-containing MCT according to
reaction solvents

| | CLA content (wt. %) | | |
|---|---|---|---|
| | Novozyrn | Lipozyrn | Lipase |
| Solvent | 435 | IM | PS-C |
| Isooctane | 51.8 ± 0.7 | 51.3 ± 0.5 | 42.1 ± 0.9 |
| n-Hexane | 56.9 ± 0.5 | 53.8 ± 1.3 | 41.4 ± 0.2 |
| Pentane | 48.6 ± 0.2 | 47.1 ± 2.3 | 29.6 ± 0.6 |
| Toluene | 40.3 ± 1.5 | 46.4 ± 0.7 | 28.3 ± 0.3 |
| Benzene | 45.4 ± 0.8 | 50.3 ± 1.4 | 16.1 ± 0.3 |
| Chloroform | 28.0 ± 0.4 | 16.2 ± 0.3 | NA |
| Acetone | 22.2 ± 0.6 | 17.4 ± 0.5 | NA |

Note:
NA means non-reactive,
*Ester form of CLA was used, and other reaction conditions are the same as Table 1.

TABLE 3
CLA content in total fatty acid composition
of CLA-containing MCT according to mix ratio
of MCT and ester form of CLA

| | Molar ratio | | | | |
|---|---|---|---|---|---|
| | 1:1 | 1:2 | 1:3 | 1:4 | 1:5 |
| Novozym | 38.4 ± 0.3 | 56.9 ± 1.7 | 67.1 ± 0.2 | 70.9 ± 0.8 | 74.8 ± 3.3 |
| 435 | | | | | |
| Lipozym | 35.0 ± 1.3 | 53.8 ± 1.3 | 64.6 ± 2.4 | 66.7 ± 0.4 | 68.1 ± 2.6 |
| IM | | | | | |
| Lipase | 25.5 ± 0.5 | 41.4 ± 1.4 | 56.5 ± 2.2 | 57.1 ± 1.9 | 64.9 ± 5.2 |
| PS-C | | | | | |

Note: The reaction was performed at 55° C. for 24 hours with 10% enzyme in hexane.

TABLE 4
CLA content in total fatty acid composition of CLA-
containing MCT according to Content of lipase enzyme

| | Lipase enzyme, % | | | |
|---|---|---|---|---|
| | 2.5 | 5.0 | 10.0 | 20.0 |
| Novozym | 44.6 ± 0.5 | 62.4 ± 1.3 | 67.1 ± 0.2 | 67.7 ± 1.3 |
| 435 | | | | |
| Lipozym | 37.0 ± 3.5 | 58.9 ± 0.4 | 64.6 ± 2.4 | 61.3 ± 0.4 |
| IM | | | | |
| Lipase | 15.9 ± 0.6 | 34.0 ± 0.9 | 56.5 ± 2.2 | 61.3 ± 2.1 |
| PS-C | | | | |

Note:
A molar ratio of tricaprylin and CLA is 1:3 by weight, and the reaction is performed at 55° C. for 24 hours.

TABLE 5
CLA content in total fatty acid composition
of CLA-containing MCT according to reaction time

| | Reaction time (hr) | | | | | | |
|---|---|---|---|---|---|---|---|
| | 1 | 2 | 3 | 6 | 12 | 24 | 36 |
| Novozym | 21.0 ± 2.2 | 37.4 ± 7.9 | 52.6 ± 0.8 | 65.2 ± 3.0 | 66.4 ± 0.3 | 67.1 ± 0.2 | 61.8 ± 0.1 |
| 435 | | | | | | | |
| Lipozym | 27.9 ± 0.5 | 40.6 ± 1.8 | 45.8 ± 0.7 | 50.3 ± 7.8 | 56.0 ± 2.2 | 64.6 ± 2.4 | 58.6 ± 2.8 |
| IM | | | | | | | |
| Lipase | 4.0 ± 0.2 | 7.7 ± 0.4 | 12.2 ± 0.6 | 21.6 ± 0.3 | 31.4 ± 1.2 | 56.5 ± 2.2 | 55.5 ± 0.1 |
| PS-C | | | | | | | |

Note: A molar ratio of tricaprylin and CLA is 1:3 by weight, and the reaction was performed at 55° C. with 10% enzyme in hexane.

TABLE 6
CLA content in total fatty acid composition
of CLA-containing MCT according to reaction temperature

| | Reaction temperature (° C.) | | | | |
|---|---|---|---|---|---|
| | 35 | 45 | 55 | 65 | 75 |
| Novozym | 59.4 ± 2.9 | 64.0 ± 3.8 | 67.1 ± 0.2 | 64.4 ± 2.2 | 63.1 ± 1.2 |
| 435 | | | | | |
| Lipozym | 58.1 ± 0.4 | 60.5 ± 4.0 | 64.6 ± 2.4 | 61.4 ± 2.8 | 60.2 ± 0.8 |
| IM | | | | | |
| Lipase | 41.9 ± 0.9 | 50.5 ± 1.9 | 56.5 ± 2.2 | 52.9 ± 0.5 | 48.6 ± 1.8 |
| PS-C | | | | | |

Note: A molar ratio of tricaprylin and CLA is 1:3 by weight, and the reaction was performed for 24 hours with 10% enzyme in hexane.

### EXAMPLE 2

### Preperation of Structured Lipid Rich in CLA at sn-2 Position

Tricaprylin as MCT was reacted in a molar ratio of 1:3 with an ester form or a free fatty acid form of CLA in 5 ml of n-hexane for 1-24 hours in a shaking water bath of 55° C. in the presence of a lipase at 5-10%. The triglyceride (TG) thus obtained was measured for the content of the CLA at sn-2 position, and the results are shown in Table 7.

TABLE 7
The content of fatty acid incorporated at sn-2
position of CLA-containing MCT

| | | The content of fatty acid | |
|---|---|---|---|
| | Incubation | at sn-2 position (wt. %) | |
| Lipase | time | Caprylic | |
| enzyme | (hr) | acid | CLA |
| Novozym | 1 | 85.3 ± 2.3 | 10.7 ± 1.4 |
| 435 | 2 | 82.0 ± 4.6 | 18.0 ± 0.8 |
| | 3 | 65.7 ± 3.1 | 30.3 ± 2.4 |
| | 6 | 40.8 ± 1.8 | 59.2 ± 3.7 |
| | 9 | 39.0 ± 1.6 | 61.0 ± 2.5 |
| | 12 | 39.4 ± 4.6 | 60.6 ± 3.3 |
| | 24 | 38.5 ± 1.7 | 61.5 ± 2.8 |
| Lipozyme | 1 | 100.0 ± 0.0 | — |
| IM | 2 | 100.0 ± 0.0 | — |
| | 3 | 100.0 ± 0.0 | — |
| | 6 | 99.2 ± 2.2 | 0.8 ± 0.3 |
| | 9 | 98.4 ± 3.5 | 1.6 ± 0.1 |
| | 12 | 93.6 ± 1.8 | 6.4 ± 0.5 |
| | 24 | 91.5 ± 5.8 | 8.5 ± 2.1 |
| Lipase | 1 | 100.0 ± 0.0 | — |
| PS-C | 2 | 100.0 ± 0.0 | — |
| | 3 | 100.0 ± 0.0 | — |
| | 6 | 100.0 ± 0.0 | — |
| | 9 | 99.6 ± 0.2 | 0.4 ± 0.2 |
| | 12 | 98.4 ± 0.5 | 1.6 ± 0.1 |
| | 24 | 95.7 ± 7.9 | 4.3 ± 0.8 |

Note:
A molar ratio of tricaprylin and CLA is 1:3 by weight, and the reaction was performed at 55° C. with 10% enzyme in hexane.

### EXAMPE 3

### Inhibition of Carcinogenesis of Mammary Gland

CLA-containing tricaprylin prepared according to the present invention was fed to rats, and its effect on development of breast cancer was observed. 3-week postnatal female Sprague-Dawley rats were divided into four groups composed of one control group and three treatment groups, with 15 rats per group. To adapt to a new environment, female rats was fed with a basic diet of AIN 76 diet comprising 20% casein, 0.3% DL-methionine, 15% corn starch, 50% sugar, 5% corn oil, 3.5% AIN mineral mixture, 1.0% AIN vitamin mixture and 0.2% choline bitartate for a week. After that, the control group was continuously fed with the basic diet while three treatment groups were fed with experimental diets for 24 weeks. In this regard, the experimental diets contained, instead of corn oil, 0.5, 2.5 and 5.0% of MCT which contained CLA in an amount of 50% in total and CLA at sn-2 position in amounts of 60, 40, 20% of the total fatty acids, respectively. 3 weeks after the adaptation, all rats were administered with one dose of 20 mg DMBA (7,12-Dimethylbenz(a)anthracene), a carcinogen specifically inducing breast cancer.

After the administration of DMBA, a latency period to the first tumor appearance was estimated in each female rat by palpation and an average latency period per group was calculated by division of the sum of the latency periods measured in a group by the number of members having mammary tumor. Tumor volume was estimated by measuring a diameter of tumor using a caliper, and calculating 4/3πr³.

TABLE 8
Latency period of mammary tumors

| | Control | Treatment group | | |
|---|---|---|---|---|
| | group | 0.5% | 2.5% | 5.0% |
| Latency | 57.8 | 86.3 | 77.7 | 80.5 |
| period (day) | | | | |

As shown in Table 8, mammary tumor appeared sooner in the control group. The treatment group with 0.5% CLA-containing MCT had 1.5 times longer latency period, compared to the control. Also, the treatment groups administered with 2.5 and 5.0% CLA-containing MCT were found to be 1.34 and 1.39 times longer in latency time, respectively, than the control.

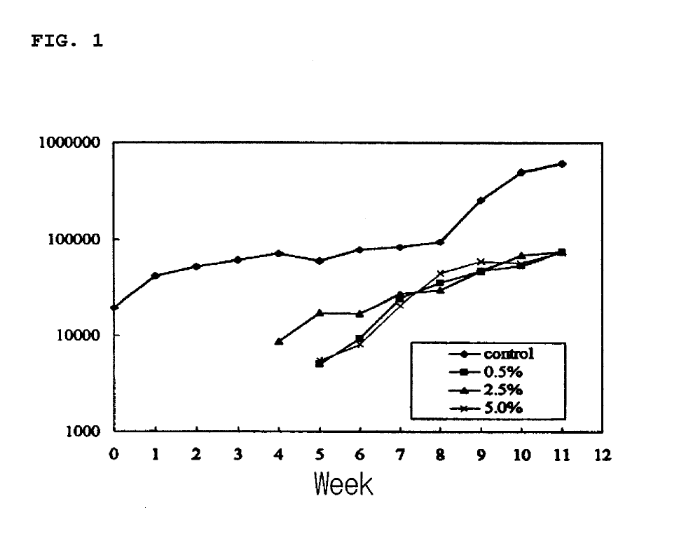

Taken together, the obtained data show that, although all of the total CLA of the CLA-containing MCT had an equal CLA content relative to total fatty acid composition and the formulated total CLA content into experimental diets was the lower at 0.5% treatment group, the higher the content of CLA at sn-2 position was, the more excellent the inhibitory activity against appearance of mammary tumor was. In addition, as shown in FIG. 1, tumors were developed at the lowest rate in the treatment group with 0.5% of CLA-containing MCT.

### EXAMPLE 4

### Inhibition Effect on Stomach Carcinogenesis

5-week postnatal male Sprague-Dawley rats were divided into four groups composed of one control group and three treatment groups, with 25 rats per group. Rats of each group were fed for 20 weeks. The control group was fed with AIN 76 diet containing sunflower oil instead of 5% corn oil. An oil product containing 60% of acid forms of CLA alkali-isomerized from sunflower oil was used, instead of corn oil, in an amount of 0.6% based on the weight of CLA in the AIN 76 diet, and the resulting diet was fed to a treatment group which was called a 0.6% CLA-FF group. Tricaprylin containing CLA in an amount of 60% in total and CLA at sn-2 position in amounts of 10 and 60% of the total fatty acids was used in amounts of 0.6% based on the weight of CLA in AIN 76 diet and the resulting modified diets were fed to treatment groups which were called 0.6% CLA-TG10 and 0.6% CLA-TG60 groups, respectively. Diets for all groups were formulated to contain lipids in an amount of 5% in total. In this regard, sunflower oil was used in the remaining amount to 5% when CLA-containing MCT was used. On day 1, MNNG (N-methyl-N′-nitro-N-nitrosoguanidine), a carcinogen inducing stomach cancer, was administrated at a dose of 200 mg/kg body weight to each rat, and from day 2, a saturated sodium chloride solution (1 ml per rat) was orally administrated four times over 2 weeks at intervals of three days, and then on the second week, MNNG was administrated once more in the same dose. After 20 weeks, forestomach and glandular stomach of each male rat were observed with naked eyes and fixed with a neutral formalin solution, and then regions showing abnormal lesion were collected and microscopically observed after preparing tissue samples with a thickness of 3-5 μm.

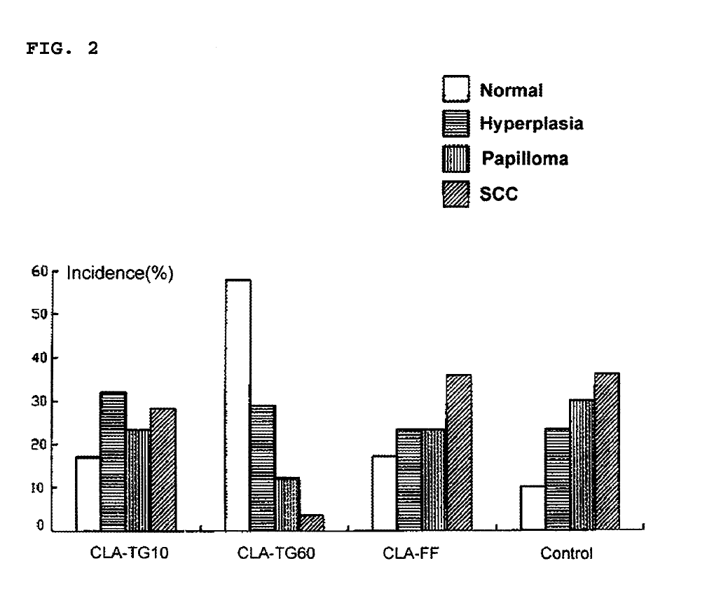

FIG. 2 provides results illustrating the incidence of normal, hyperplasia, papilloma and squamous cell carcinoma (SCC) according to contents of CLA. As a result of statistical analysis according to frequency of hyperplasia, papilloma and squamous cell carcinoma (SCC), the incidence of stomach cancer in the 0.6% CLA-TG60 group was significantly inhibited in comparison with in both the control group and 0.6% CLA-TG10 group, and the incidence of normal tissue in the 0.6% CLA-TG60 group was 56%, which was higher than in the other groups. Accordingly, CLA-containing MCT having higher content of CLA at sn-2 position showed more excellent inhibition effect on incidence of stomach cancer. Similar inhibition effects were detected in between 0.6% CLA-TG10 and 0.6% CLA-FF groups.

### INDUSTRIAL APPLICABILITY

As described above, the present invention relates to the method for preparing a CLA-containing triacylglycerol by transesterification of a mixture of MCT and CLA composed of acid or ester form of cis-9, trans-11 and trans-10, cis-12 octadecadienoic acids, using lipase, and particularly, the method can control and enhance CLA content at the sn-2 position of TG. When a CLA-containing TG of the present invention is administered to mammals, inhibition effects on appearance of mammary tumor, stomach cancer and the like can be obtained, and moreover, a CLA-containing TG having high CLA content at the sn-2 position shows more excellent inhibition effects. In addition, even when being administered at a small amount, innate nutritional properties of MCT can supply calories, and CLA can express its biological activities, such as inhibition of carcinogenesis and reduction of accumulation of body fat. And also, a CLA-containing TG of the present invention is useful as a therapeutic material because general MCT is, at present, used as a rapid calorie supply source during medical treatment.

1. A conjugated linoleic acid-containing synthesized acylglyceride having biological activities, represented by the following general formula I: 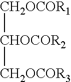
wherein R₁, R₂ and R₃, which may be the same or different, are akyl chains selected from the group consisting of alkyl moieties of medium chain fatty acids of C₆ to C₁₀ and long chain fatty acids of C_{18:2}, said C_{18:2} being a conjugated linoleic acid (CLA) selected from among cis-9, trans-11 octadecadienoic acid and trans-10, cis-12 octadecadienoic acid.

2. The linoleic acid-containing synthesized acylglyceride as set forth in claim 1, wherein the medium chain fatty acid is selected from the group consisting of caproic acid (C_{6:0}), caprylic acid (C_{8:0}), capric acid (C_{10:0}), and mixtures thereof.

3. The linoleic acid-containing synthesized acylglyceride as set forth in claim 1, wherein content of the cis-9, trans-11 and trans-10, cis-12 octadecadienoic acids is at least 15% by weight in total fatty acid composition.

4. The CLA-containing structured lipid as set forth in claim 1, wherein content of the cis-9, trans-11 and trans-10, cis-12 octadecadienoic acids at sn-2 position is at least 5% in total fatty acid composition.

5. A method for preparing a conjugated linoleic acid-containing synthesized acylglyceride, in which free or ester forms of conjugated linoleic acid are reacted in a molar ratio of 1:1˜1:5 with acylglyceride at 35˜75° C. for 1˜36 hours on an immobilized phase of a lipase in a solvent, said lipase being present in an amount of 2.5˜20% by weight of the total weight of the conjugated linoleic acid and the acylglyceride.

6. The method as set forth in claim 5, wherein the solvent is selected from the group consisting of isooctane, n-hexane, pentane, toluene, benzene, chloroform, acetone, and mixtures thereof.

7. The method as set forth in claim 5, wherein the immobilized lipase is an extracellular enzyme and is selected from the group consisting of lipases obtained from Pseudomonas fluorecens, Candida rugosa, Aspergillus niger, Pseudomonas cepacia, Rhizomucor miebei, Candida antartica and combinations thereof.

8. Use of conjugated linoleic acid-containing structured acylglyceride in feedstuffs, foods, and medical treatment, whereby biological activities of rapid calorie supply, carcinogenesis inhibition, and body fat reduction can be obtained in animals and humans.

9. The use as set forth in claim 8, wherein the conjugated linoleic acid-containing structured acylglyceride is added in an amount of at least 0.1% by weight to feedstuff or food.
